(19) United States
(12) Patent Application Publication
(10) Pub. No.: US 2010/0047434 A1
(43) Pub. Date: Feb. 25, 2010

(54) FABRICATION OF MONOLITHIC ZONES ON POROUS SCAFFOLD

(75) Inventor: Mukesh Kumar, Warsaw, IN (US)

Correspondence Address: HARNESS, DICKEY & PIERCE, P.L.C., P.O. BOX 828, BLOOMFIELD HILLS, MI, 48303, US

(73) Assignee: Biomet Manufacturing Corp., Warsaw, IN (US)

(21) Appl. No.: 12/195,770

(22) Filed: Aug. 21, 2008

(51) Int. Cl. A61L 31/08; A61F 2/02

(52) U.S. Cl. 427/2.1; 623/11.11

(57) ABSTRACT

Processes for preparing medical devices comprising building, by confocally fed, laser-assisted microdeposition, a monolithic biocompatible ceramic coating on a surface of a porous or porogenic, biocompatible substrate; medical devices prepared thereby; and kits comprising them.

### BACKGROUND

The present disclosure relates to medical implants having a porous body or core and a laser-deposited, monolithic coating on at least one surface thereof.

In the growing field of medical devices, there is a continued need to provide lightweight orthopedic implants having enhanced strength, such as porous implants having enhanced surface strength.

One technique that has been used to provide improved medical implants is to coat a substrate with a film of a biocompatible target. However, coating processes among those in the art may not ensure a strong substrate-to-coating bond, and are generally not useful for providing a precisely shaped coat. Thus, it would be desirable to provide a method for forming improved precision implants and improved lightweight implants by use of a direct, additive deposition process.

### SUMMARY

The present technology provides medical devices comprising a porous substrate coated with a biocompatible coating. In various embodiments, methods are provided for preparing a medical device that comprises a porous biocompatible substrate and a biocompatible monolithic coating on a surface thereof, the process comprising: (A) providing (1) a porous biocompatible substrate comprising a porous biocompatible metallic, ceramic, or glass-ceramic material; (2) a supply of particulate raw material capable of being melt-deposited to form a biocompatible ceramic or glass-ceramic layer upon the porous substrate surface; (3) a supply of carrier gas; and (4) a computer-programmable, automated, laser-assisted microdeposition (LAM) device having at least one integrated deposition head, the head both comprising a laser and having at least two particle delivery nozzles, each of which is so positioned as to be capable of directing a stream of the gas carrier confocally to the focal point of the laser; (B) placing a portion of said surface of said substrate in contact with the laser beam at its focal point; (C) mixing particulate raw material (A2) with carrier gas (A3) to form a particle stream; (D) feeding the particle stream into each of at least two of said delivery nozzles of the deposition head, whereupon the laser at least partially melts the particulate raw material delivered to the focal point; (E) cooling the particulate raw material to form a solid deposit of biocompatible ceramic or glass-ceramic material on said portion of said surface substrate; and (F) moving the substrate relative to the laser beam to direct the laser beam focal point, at least once, across the remainder of said surface in a predetermined pattern, while performing said mixing, feeding and cooling steps, by which a layer of biocompatible ceramic or glass-ceramic material is deposited on substantially the entirety of said surface, at least the final deposited layer thereof being a contiguous layer, thereby forming a biocompatible, monolithic ceramic or glass-ceramic coating on said surface,

### DETAILED DESCRIPTION

The following description of technology is merely exemplary in nature of the subject matter, manufacture and use of one or more inventions, and is not intended to limit the scope, application, or uses of any specific invention claimed in this application or in such other applications as may be filed claiming priority to this application, or patents issuing therefrom. The following definitions and non-limiting guidelines must be considered in reviewing the description of the technology set forth herein.

The headings (such as “Introduction” and “Summary”) and sub-headings used herein are intended only for general organization of topics within the present disclosure, and are not intended to limit the disclosure of the technology or any aspect thereof. In particular, subject matter disclosed in the “Introduction” may include novel technology and may not constitute a recitation of prior art. Subject matter disclosed in the “Summary” is not an exhaustive or complete disclosure of the entire scope of the technology or any embodiments thereof. Classification or discussion of a material within a section of this specification as having a particular utility is made for convenience, and no inference should be drawn that the material must necessarily or solely function in accordance with its classification herein when it is used in any given composition.

The citation of references herein does not constitute an admission that those references are prior art or have any relevance to the patentability of the technology disclosed herein. All references cited in the “Description” section of this specification are hereby incorporated by reference in their entirety. However, in the event of a conflict between the terms of an incorporated reference and the terms of this disclosure, the terms of this disclosure should be followed.

The description and specific examples, while indicating embodiments of the technology, are intended for purposes of illustration only and are not intended to limit the scope of the technology. Moreover, recitation of multiple embodiments having stated features is not intended to exclude other embodiments having additional features, or other embodiments incorporating different combinations of the stated features. Specific examples are provided for illustrative purposes of how to make and use the compositions and methods of this technology and, unless explicitly stated otherwise, are not intended to be a representation that given embodiments of this technology have, or have not, been made or tested.

As referred to herein, all compositional percentages are by weight of the total composition, unless otherwise specified. As used herein, the word “include,” and its variants, is intended to be non-limiting, such that recitation of items in a list is not to the exclusion of other like items that may also be useful in the materials, compositions, devices, and methods of this technology.

The present technology provides methods for coating a porous substrate with a biocompatible coating. In various embodiments, the substrate comprises a metallic, ceramic or glass-ceramic material. In various embodiments, the coating comprises a ceramic or glass ceramic material.

### Ceramic Materials for Deposition

The ceramic, glass, or glass ceramic coating can be made up of one or more layers of any biocompatible ceramic, glass, or glass ceramic material, or a combination thereof. Examples of useful coating material types include the biocompatible ceramic-, glass-, or glass ceramic carbides, nitrides, borides, silicides, and oxides. The can be metal or non-metal materials; for example, non-metal materials can be diamond, diamond-like carbon, boron carbide, or silicon nitride. In some embodiments in which a metal boride, carbide, nitride, silicide, and/or oxide is deposited, the metal can be calcium, titanium, zirconium, hafnium, vanadium, niobium, tantalum, chromium, molybdenum, tungsten, or aluminum, or a combination thereof, although nickel, iron, alkali metal(s), non-calcium alkaline earth metal(s), or in some embodiments, gold, silver, copper, palladium, platinum, bismuth, cobalt, tin, or zinc, can be present.

Examples of useful carbide materials include silicon carbide, titanium carbide, tungsten carbide, vanadium carbide, chromium carbide, molybdenum carbide, tantalum carbide, niobium carbide, zirconium carbide, boron carbide, hafnium carbide, mixed carbides (e.g., aluminum/boron carbide, cobalt/tungsten carbide, nickel iron/tungsten carbide, and nickel molybdenum/titanium carbide), and combinations of such carbides.

Examples of useful nitride materials include silicon nitride, boron nitride, carbon nitride, aluminum nitride, titanium nitride, titanium aluminum nitride, titanium carbon nitride (TiCN), tantalum nitride, chromium nitride, zirconium nitride, silicon carbon nitride, boron nitride, and hafnium nitride, mixed nitrides (e.g., carbonitrides, such as titanium carbonitride), and combinations of such nitrides.

Examples of useful boride materials include TiB₂, ZrB₂, TaB₂, MnB₂, MoB₂, VB₂, CrB₂, HfB₂, NbB₂, W₂B₅, Mo₂B₅, AlB₂, mixed borides, and combinations of such borides. Examples of useful silicide materials include molybdenum disilicide, tantalum silicide, mixed silicides, and combinations of such silicides.

Examples of useful oxide materials include calcium oxides, calcium phosphates, alumina, zirconia, titania, borates, silica, silicates (e.g., aluminum silicate), titanates (e.g., aluminum titanate, barium titanate), magnesia, titanium/aluminum oxide, aluminum/aluminum oxide, calcium sulfates, niobium oxide, lithium niobate, mixed oxides, and combinations of such oxides. Among the calcium phosphates, useful examples of these include: hydroxyapatites including CaiO(PO₄)₆(OH)₂, strontium hydroxyapatite, calcium-strontium hydroxyapatite, halo-hydroxyapatites, and the like, and amorphous forms thereof; tricalcium phosphate (Ca₃(PO₄)₂); tetracalcium phosphate (Ca₄P₂O₉), and combinations thereof, e.g., calcium phosphate glass containing a calcium phosphate ceramic phase, glass-ceramics, and so forth (e.g., Na₂O.CaO.P₂O₅.SiO₂ or 21B₂O₃.8CaO.9Na₂O.5P₂O₅ glass ceramics; SiO₂.P₂O₅.CaO.MgO glasses).

In addition to a biocompatible ceramic, glass or glass ceramic, the coating may also comprise a metal. Such metals may be the same or different as metals that, in some embodiments, are used in the substrate. Metals useful in the coating include titanium, titanium alloys (e.g., Ti₆Al₄V), Co—Cr alloys (e.g., Co—Cr—Mo alloys), stainless steel (e.g., 316L), and alloys thereof with other metal(s). Any combination of metal (alloy) can be used with any ceramic to create a hard and wear resistant monolithic bearing surface. The wear resistant monolithic bearing surface would be more like a “cermet” which is a composite material composed of ceramic and metal. Such a composite of metal-ceramic may have optimal properties of both a ceramic, such as hardness, and those of a metal. In some embodiments, the metal may function as a binder to hold the ceramic particles. The particles may be present at a level of about 60 to 80% by volume in such ceramic metal composites.

The layers of a given coating can be the same or different. A series of layers can be deposited so as to improve adhesion of a final material to the substrate by providing intermediate layers of mixed composition or of a material having better adhesion to the substrate or sublayer and the overlayer. See, e.g., B. Bell et al., Pulsed laser deposition of hydroxyapatite-diamondlike carbon multilayer films and their adhesion aspects, J. Adhesion Sci. & Tech. 20(2-3):221-31 (2006). In some embodiments, one or more layers comprising a metal may be deposited in addition to one or more layers comprising a ceramic or glass ceramic material.

The powdered starting materials to be delivered to the laser focal point for microdeposition of the ceramic, glass, or glass ceramic will comprise elements to be deposited, and can have substantially the same elemental composition as that of the ceramic, glass, or glass ceramic to be deposited. In some embodiments, the composition of the starting material can be the same as that of the intended deposit. In some embodiments, the carrier gas itself can comprise a gas or vapor starting material for deposition. In various embodiments, two or more different starting material powders (and/or gases) can be delivered, by different streams, to the laser focal point; or a mixture of different powders (and/or gases) can be delivered by each of the streams.

For example: a silicon carbide deposit can be formed from a tetramethylsilane (TMS) gas precursor or from silicon carbide powder; a silicon nitride deposit can be formed from a combination of TMS and ammonia gas precursors; a carbon deposit can be formed from acetylene; or a hydroxyapatite deposit can be formed from CaO and CaHPO₄.2H₂O or from hydroxyapatite powder.

The particles of the starting material powder can be about 150 μm or smaller in average diameter, or less than or about 100 or 50 or 20 or 10 μm; the particles can be about 50 nm or greater in average diameter, or about or at least: 100 nm or 1 μm or 5 or 10 μm. In some embodiments, the average particle size can be from about 1 to 50 gum; in some embodiments, the average particle size can be from about 50 nm to 10 μm.

### Carrier Gas

The carrier gas can be an inert or reactive gas or gas mixture. As used herein, “reactive gas” refers to a gas that is capable of reacting, pursuant to contact with the laser focal point, to provide useful material to the deposit. In embodiments using a reactive gas, the gas can be selected according to the ceramic to be deposited. Thus, for deposition of an oxide ceramic, the carrier gas can comprise oxygen, e.g., molecular oxygen, or an oxygen-containing gas, e.g., water or hydrogen peroxide, or a combination thereof with an inert gas. Similarly, for deposition of a nitride ceramic, the carrier gas can comprise nitrogen, e.g., in the form of molecular nitrogen or ammonia, or a combination of a nitrogen-containing gas with an inert gas. Likewise, for deposition of a carbide ceramic, the carrier gas can comprise carbon, e.g., in the form of carbon dioxide or methane, or a combination of a carbon-containing gas with an inert gas. Combinations of reactive carrier gases (i.e. true gases or vapors) can be used for any given ceramic type. Inert gases include the noble gases, among which He, Ne, Ar, Kr, and mixtures thereof are particularly useful; Ar is commonly used.

In various embodiments, an inert gas alone can be used. In various embodiments in which a reactive gas is used, it can be up to or about 50%, 30%, 25%, 20%, 15%, 10%, or 5% by volume of the carrier gas; it can be about or at least 1%, 5%, or 10% thereof. In various embodiments in which hydroxyapatite is deposited, the carrier gas can be a combination of an oxygen-containing gas (e.g., water or hydrogen peroxide) with an inert gas, e.g., Ar. The carrier gas, or one or more component gases thereof, such as a reactive gas thereof, can be supplied as a cooled gas, a room/ambient temperature gas, or a heated gas. Such a gas can be heated, e.g., from about 25 to about 200° C., to about 100° C., or to about 50° C.

In various embodiments, the reaction chamber for LAM can be held under reduced pressure, e.g., under vacuum conditions such as about 0.1-10 mTorr. However, either the gas in the reduced pressure chamber, or the carrier gas, or both, can contain an inert gas, reactive gas, or combination thereof; or after vacuum evacuation, the chamber can be filled therewith to a desired pressure, e.g., 1-760 Torr. Where a reactive gas is used, its partial pressure, in various embodiments, can be about or at least 1, 10, 100, or 200 mTorr, can be up to or about 1000, 800, 600, 500, 400, 300, or 250 mTorr, or can range from about 1-1000, 10-800, 100-600, or 200-500 mTorr.

### Substrate Materials

A substrate material for use in an implant hereof can be any porous or porogenic biocompatible implant material capable of weight bearing that is inorganic. Useful examples of such materials include metals, ceramic, and composites thereof with one another and/or with a further material, such as carbon. Specific examples of useful substrate materials include titanium, titanium alloys (e.g., Ti₆Al₄V), Co—Cr alloys (e.g., Co—Cr—Mo alloys), stainless steel (e.g., 316L), alloys thereof with other metal(s); ceramics; and composites, e.g., ceramic-metal composites or composites with carbon.

In some embodiments, the substrate upon which deposition is to take place can contain an organic compound or compounds, whether functioning as a porogen, matrix material, adhesive, or as a filler or reinforcing element in a composite material. Thus, in various embodiments, the substrate can be non-porous, but containing porogens or other elements that are either biodegradable in vivo so as to be capable of forming a porous substrate in vivo or degradable by pre-implantation treatment after deposition of the coating.

In various embodiments, the porous material can comprise pores of about 1-500 μm or 50-500 μm average diameter. The pores can be present as an interconnected network that permits permeation across the network; in various embodiments, the pore network or networks can be located in one or more zones of the substrate, and such a pore network can be internal only or surface-accessible; in various embodiments, such a network can be surface-accessible. The network can be present during deposition of the coating, can be exposed after deposition and before implantation, or if biodegradable in vivo can be exposed after implantation.

### Laser Deposition Devices

Laser-assisted microdeposition (LAM) as used herein refers to a process of deposition employing laser(s) to melt or sinter powdered starting materials confocally delivered to the focal point of the laser at or on the surface upon which the deposit is made. The melting or sintering results in formation of the deposit on the surface. In various embodiments, LAM is synonymous with laser-engineered net shaping, also called laser-engineered near-net shaping; thus, LAM may be performed using, e.g., a LENS system (Nd:YAG or fiber laser engineered net shaping system, available from Optomec Inc., Albuquerque, N. Mex., USA), a LASFORM system (CO₂ laser assisted forming system, available from AeroMet Corp., Eden Prairie, Minn., USA); or another similar system.

The laser employed can be, e.g., a Nd:YAG laser, an argon, helium, or neon laser, a carbon dioxide laser, a fiber laser, or a KrF excimer laser; the laser is operated at a wavelength (e.g., 355 nm) and energy density useful to provide melting or sintering conditions for the powdered starting materials at the substrate surface. The laser can be operated in continuous or pulsed mode.

The laser thus melts or sinters materials delivered by the converging powder streams so as to enact microdeposition. The movement of the substrate, according to a predetermined pattern (e.g., a raster pattern or vector pattern) permits movement of the point or zone of microdeposition, thereby extending the microdeposit into a straight or curved line; a series of, e.g., substantially parallel or substantially concentric lines forms the pattern by which the surface area is covered with a deposited layer.

Alignment of the laser raster pattern can be performed by use of a laser or laser array of low-energy light, i.e. not capable of melting or sintering the substrate, to pre-scan the surface to be coated prior to the coating process. One or more scans of the substrate surface can be performed and the optimal direction or directions for line building can be determined. For example, a detector or detector array can be used to receive reflected light and, based on the detected light, a visual or automated process can be employed to identify therefrom a line build pattern that can maximize layer-to-surface contact. Techniques such as described, e.g., in U.S. Pat. No. 5,343,548 for matching a laser array to a fiber array can be adapted for this purpose so as to enhance or optimize a line build pattern relative to an arrangement of pore-defining walls on a substrate surface hereof, whether the pore spaces thereof are empty or porogen-containing.

One feature of a laser-assisted microdeposition (LAM) system for use herein is confocal delivery of powdered starting material to the focal point of the laser, by at least two streams. In some embodiments hereof, for these streams, Laser Particle Guidance (LPG) can be utilized to obtain resolution on the order of about 0.1-10 μm line width, or Flow Guidance (FG) can be utilized to obtain resolution on the order of about 10-25 μm line width. For such embodiments, the starting material powder particle size can be about 10 μm or smaller in average diameter, or about 5, or 2, or 1 μm or smaller; particle size can be about 50 nm or larger, or about 100 nm or larger. See, e.g., M. J. Renn et al., Laser Guidance and Trapping of Mesoscale Particles in Hollow-Core Optical Fibers, Phys. Rev. Lett. 82(7):1574-77 (February 1999); and M. J. Renn et al., J. Vac. Sci. & Tech. B: Microelec. & Nano. Str. 16(6):3859-63 (November 1998).

A LAM system can be operated according to manufacturer's instructions. Typically, laser deposition conditions can include use of: a laser fluence of about 1-10 J/cm², or about 2-5, about 3-4.5, or about 4 J/cm², and use of a 50 W-20 kW laser operated in a 300-600 W or 400-450 W power range. Substrate pre-heating can be used in various embodiments, such as heating to an elevated temperature in the range of about 25-600° C., or about 25-400° C.

In the case of calcium phosphate ceramics, a system can be operated under process conditions similar to those utilized in, e.g.: J. L. Arias et al., Stoichiometric transfer in pulsed laser deposition of hydroxylapatite, Appl. Surf Sci. 154-155:434-38 (2000); Q. Bao et al., Pulsed laser deposition of hydroxyapatite thin films under Ar atmosphere, Matls. Sci. & Eng.:A 429(1-2):25-29 (August 2006) (depositing HA onto Ti); B. Bell et al., Pulsed laser deposition of hydroxyapatite-diamondlike carbon multilayer films and their adhesion aspects, J. Adhesion Sci. & Tech. 20(2-3):221-31 (2006); O. Blind et al., Characterization of hydroxyapatite films obtained by pulsed-laser deposition on Ti and Ti-6Al-4V substrates, Dent. Matls. 21(11):1071-24 (November 2005); L. Clèries et al., Application of dissolution experiments to characterize the structure of pulsed laser-deposited calcium phosphate coatings, Biomaterials 20:1401-05 (1999); C. M. Cotell et al., Pulsed laser deposition of hydroxyapatite thin films on Ti6Al4V, J. Appl. Biomaterials 3:87-93 (1992); or M. Jelinek et al., Effect of processing parameters on the properties of hydroxylapatite films grown by pulsed laser deposition, Thin Solid Films 257:125-29 (1995). Also see: W. Hofmeister et al., Investigating Solidification with the Laser-Engineered Net Shaping (LENS) Process, J Minerals, Metals & Matls [JOM] 51(7) (July 1999); and J. Kummailil, Process Models for Laser Engineered Net Shaping (May 2004) (Ph.D. Dissertation, Worcester Polytechnic Institute). Thus, in some embodiments, the temperature of the substrate and/or the energy delivered to the laser focal point can be greater during deposition of the first layer or first few layers in order to obtain substrate melt and/or deposit melt conditions, and then reduced during deposition of subsequent layers.

In embodiments in which more than one layer is to be deposited to form the surface coating, after completion of a given layer, the distance between the laser and the treated surface is increased such that the focal point of the laser will coincide with the surface of the most recently deposited layer.

In various embodiments, the coating deposited on the substrate surface can be from about 1 μm to about 10 mm in thickness. At least the final layer of the coating will be monolithic; in some embodiments, about or at least the uppermost 50% of layers of the coating can be monolithic.

As the laser melt operation is normally conducted in an inert atmosphere, it is possible that certain ceramics like alumina and silicon nitride may lose some of its oxygen or nitrogen to the atmosphere resulting in an anionic deficient system. This can be addressed by reheating these deficient systems in suitable atmosphere e.g. alumina parts under argon with oxygen partial pressure and silicon nitride parts under argon with nitrogen partial pressure.

In order to reduce any voids between layers (or within any individual layer), it is possible to HIP the part where the part is exposed to high pressure and high temperature. Such techniques include common processing techniques routinely used in removing voids from cast metal parts and structural parts.

### Coordinate Deposition

In various embodiments hereof, a layer can be formed on at least one surface the substrate, by coordinate deposition. In various embodiments, the coating may be applied to only part of the surface, or to substantially the entire surface of the substrate. As used herein, phrases such as “coordinate depositing on” and “deposition coordinate to” the surface arrangement of pores at the surface of a given porous substrate indicates that deposition of a layer takes place in substantially parallel or substantially concentric lines positioned, and optionally sized by width, so as to increase the contact area between the layer and the pore-defining walls of the substrate, resulting in a contact area that is greater than the average contact area typically obtained by non-coordinate deposition. This criterion is particularly useful for the first layer, or first few layers to be deposited on a porous substrate. The raster or vector pattern used in forming such a layer can be referred to as a “coordinate deposition pattern.”

In a raster or vector pattern for the microdeposition of the lines of a build layer in some coordinate deposition embodiments hereof, the average center-to-center distance between lines of a coordinately deposited layer can be equal to the value, or to a multiple of the value, of the average wall-to-wall distance, measured transversely across pores, from the center of one pore wall to the center of the opposite pore wall.

Where the pores are generally regular in cross section, as circles or squares, if the pores are oriented randomly at the surface, then the average wall-to-wall distance can be determined in any one direction across the surface.

Where the pores are generally oblong in cross section, as ellipses or rectangles, if the pores are oriented randomly at the surface, then the average wall-to-wall distance can itself be an average of an average of distances determined in any two or more directions across the surface.

Where the pores of any type are arranged in a substantially regular pattern at the surface, the average wall-to-wall distance can be determined from the concomitant substantially regular arrangement of pore walls. Thus, e.g., where the pores are generally oblong in cross section and are oriented at the surface with their main axes generally parallel, the average wall-to-wall distance can be determined by measuring pore wall center-to-center distance across either the major axes or the minor axes thereof. Similar principles apply to determine useful average wall-to-wall distances for use with other pore arrangements.

In various embodiments, in which surface pores of a substrate are spaced further apart, such that the pore-defining walls present at the surface are wider, the rows of a coordinately deposited layer can be wider than those used in embodiments in which pore-defining walls present at the surface of a substrate are narrower.

Where pores are arranged according to a continuously or discontinuously changing geometry, coordinately deposited rows can be spaced according to a concomitantly changing arrangement of pore walls.

Among the first and subsequent few layers, or the first group and subsequent few groups of layers, the orientation of rows can differ; for example, the rows of a second layer can be deposited in a direction transverse (oriented about 90°) to that of the rows of a first layer, or diagonal thereto (e.g., oriented about 15°, 30°, 45°, 60°, or 75° thereto). In various embodiments, such a different orientation of the rows of a subsequent layer can constitute a second coordinate deposition arrangement that is likewise determined from the surface arrangement of substrate pores.

In embodiments of a coating in which at least one layer thereof is coordinately deposited on a porous substrate, the coating can be referred to as, e.g., a “coordinately deposited” coating. In various embodiments, at least two, or at least three layers will be coordinately deposited; in some embodiments, at least or about 20%, 30%, 40%, or 50% of the layers of a coating will be coordinately deposited.

### Uses

The coated substrates hereof can be useful in or as prosthetic bone substitutes, implantable splints, dental implants, components thereof, and similar medical devices. In various embodiments, the surface of the substrate is an articulating surface, i.e., the surface of an implant which articulates with a second surface. The second surface may be on a bone or other body structure, or on a second implant component. For example, the surface of the substrate may be part of a hip, knee, shoulder or elbow implant, articulating with either a natural bone structure or other implant. The present technology also provides a kit comprising a coated substrate with instructions for use thereof in or as such a medical device, e.g., instructions for implantation or for pre-implantation treatment steps such as (1) hydration with neat or patient-autologous fluid, (2) removal of porogens, or (3) loading of porogens with bioactive agents such as bone, cartilage, or other tissue growth or differentiation factors, hormones, antibiotics, anti-rejections medicaments and the like.

The materials, compositions and methods of this technology are exemplified in the following non-limiting examples.

### EXAMPLE 1

A stream of alumina is injected with carrier gas of argon onto a focused laser beam on the surface of porous Ti₆Al₄V construct. The localized heat of the laser melts the ceramic on the surface of the porous Ti₆Al₄V construct. This molten mass fuses with the porous Ti₆AM₄V construct and adjoining alumina particle. The particle size of the powder is controlled to less than 0.25 microns and the particle concentration in the carrier gas stream is so chosen to achieve a continuous zone of ceramic structure. The deposited layers may be repeated to build thicker monolithic zones with little or no porosity.

### EXAMPLE 2

In another example, the method of Example 1 is followed but using silicon nitride or silicon carbide powder. The particle size is kept at less than 0.25 microns and the particle concentration in the carrier gas stream is so chosen to achieve a continuous zone of ceramic structure. The deposited layers may be repeated to build thicker monolithic zones with little or no porosity.

### EXAMPLE 3

The method of Example 1 is modified where the carrier gas has Co—Cr—Mo particles with Alumina particles. The ratio of the two materials is so chosen to provide a bearing surface where the Co—Cr—Mo has dispersed alumina. The resulting structure is harder and a less wearing material compared to Co—Cr—Mo. Alternatively, the alloy is replaced with tungsten (or other hard) alloy and silicon nitride ceramic used instead of alumina. The deposited layers may be repeated to build thicker monolithic zones with little or no porosity.

These materials made in Examples 1, 2 and 3, and modifications or combinations thereof, may be ground, honed and polished to create smooth surfaces with surface roughness less than 100 nanometers to generate articulating surfaces. In various embodiments, the surface roughness is less than 50 nanometers, or less than 5 nanometers. The materials made in Examples 1, 2 and 3, and modifications or combinations may also be made rough, optionally with layers of calcium phosphate materials, to allow increased biocompatibility.

The embodiments and the examples described herein are exemplary and not intended to be limiting in describing the full scope of compositions and methods of the present technology. Equivalent changes, modifications and variations of some embodiments, materials, compositions and methods can be made within the scope of the present technology, with substantially similar results.

What is claimed is:

1. A process for preparing a medical device that comprises a porous biocompatible substrate and a biocompatible monolithic coating on a surface thereof, the process comprising:
(A) providing
(1) a porous biocompatible substrate comprising a porous biocompatible metallic, ceramic, or glass-ceramic material;
(2) a supply of particulate raw material capable of being melt-deposited to form a biocompatible ceramic or glass-ceramic layer upon the porous substrate surface;
(3) a supply of carrier gas; and
(4) a computer-programmable, automated, laser-assisted microdeposition (LAM) device having at least one integrated deposition head, the head both comprising a laser and having at least two particle delivery nozzles, each of which is so positioned as to be capable of directing a stream of the gas carrier confocally to the focal point of the laser;
(B) placing a portion of said surface of said substrate in contact with the laser beam at its focal point;
(C) mixing particulate raw material (A2) with carrier gas (A3) to form a particle stream;
(D) feeding the particle stream into each of at least two of said delivery nozzles of the deposition head, whereupon the laser at least partially melts the particulate raw material delivered to the focal point;
(E) cooling the particulate raw material to form a solid deposit of biocompatible ceramic or glass-ceramic material on said portion of said surface substrate; and
(F) moving the substrate relative to the laser beam to direct the laser beam focal point, at least once, across the remainder of said surface in a predetermined pattern, while performing said mixing, feeding and cooling steps, by which a layer of biocompatible ceramic or glass-ceramic material is deposited on substantially the entirety of said surface, at least the final deposited layer thereof being a contiguous layer, thereby forming a biocompatible, monolithic ceramic or glass-ceramic coating on said surface.

2. The process according to claim 1, further comprising machining the device or component.

3. The process according to claim 1, wherein said substrate is moved across the surface area more than once to deposit more than one layer on the surface to form the coating.

4. The process according to claim 3, wherein all the layers are contiguous layers.

5. The process according to claim 3, wherein the composition of the particle stream used to form each of a sequence of layers is changed between layers or between groups of layers so as to from a chemical or physical gradient among the layers.

6. The process according to claim 5, wherein the particle stream composition is changed by successively increasing the volume/volume particle concentration of the particle stream.

7. The process according to claim 5, wherein the particle stream composition is changed by successively increasing the percent content of at least one component, and fewer than all components, in a multi-component particle stream.

8. The process according to claim 1, wherein the particles have an average diameter of about 0.1 to about 100 microns.

9. The process according to claim 1, wherein the porous substrate comprises a porous biocompatible metallic titanium, titanium alloy, cobalt-chromium alloy, tantalum, or stainless steel.

10. The process according to claim 1, wherein the porous substrate comprises a porous biocompatible titanium alloy.

11. The process according to claim 10, wherein the titanium alloy further comprises nickel, aluminum, vanadium, niobium, tantalum, zirconium, molybdenum, or a combination thereof.

12. The process according to claim 10, wherein the titanium alloy is a TiAl₆V₄ alloy.

13. The process according to claim 1, wherein the surface of said substrate is an articulating surface.

14. The process according to claim 1, wherein the particulate raw material is capable of being melt-deposited to form a biocompatible ceramic or glass-ceramic layer of a porcelain, calcium phosphate compound, titania, alumina, calcium oxide, silicon carbide, silicon nitride, or combination thereof.

15. The process of claim 14, wherein the particulate raw material comprises titanium, titanium alloys (e.g., Ti₆Al₄V), Co—Cr alloys (e.g., Co—Cr—Mo alloys), stainless steel (e.g., 316L), and alloys comprising such metals.

16. The process according to claim 1, wherein the particulate raw material is capable of being melt-deposited to form a biocompatible layer of a hydroxyapatite compound.

17. The process according to claim 1, wherein for at least the first layer deposited on the substrate, the predetermined pattern of step (E) is a coordinate deposition pattern.

18. The process according to claim 1, wherein said process further comprises determining a coordinate deposition pattern prior to step (B).

19. A medical device or medical device component comprising a porous biocompatible metallic, ceramic, or glass-ceramic substrate and a confocally-laser-deposited coating of biocompatible monolithic ceramic, or glass-ceramic coating on a surface thereof.

20. A medical device according to claim 19, wherein the coating is non-laminated and reinforces the scaffolding of the porous substrate.

21. A medical device according to claim 19, wherein the surface is an articulating surface.

22. The device or component according to claim 19, wherein the porous substrate comprises a porous biocompatible titanium alloy.

23. The device or component according to claim 19, wherein the coating comprises a biocompatible ceramic or glass-ceramic layer of a porcelain, calcium phosphate compound, titania, alumina, calcium oxide, silicon carbide, silicon nitride, or combination thereof.

24. The device or component according to claim 23, wherein the coating further comprises titanium, titanium alloys (e.g., Ti₆Al₄V), Co—Cr alloys (e.g., Co—Cr—Mo alloys), stainless steel (e.g., 316L), and alloys comprising such metals.

25. The medical device according to claim 19, wherein the coating comprises at least one coordinately deposited layer.
